(19) United States
(12) Patent Application Publication
(10) Pub. No.: US 2026/0096587 A1
(43) Pub. Date: Apr. 9, 2026

(54) COMPOSITIONS AND METHODS FOR ACTIVATING GLP-1 SECRETION

(71) Applicant: LifeVantage Corporation, Lehi, UT (US)

(72) Inventors: Elisa Diane BARNES, Draper, UT (US); Christina Beer, Salt Lake City, UT (US); Clinton T. Horton, Salt Lake City, UT (US); Robert A. Carr, American Fork, UT (US)

(21) Appl. No.: 19/056,117

(22) Filed: Feb. 18, 2025

Related U.S. Application Data
(63) Continuation-in-part of application No. 18/910,816, filed on Oct. 9, 2024.

(51) Int. Cl. A23L 33/00; A61K 31/23; A61K 31/718; A61K 36/06; A61K 36/45; A61K 36/752; A61K 36/87

(52) U.S. Cl. CPC A23L 33/40; A23L 33/30; A61K 31/23; A61K 31/718; A61K 36/06; A61K 36/45; A61K 36/752; A61K 36/87

(57) ABSTRACT

Nutritional supplements and compositions for activating the GLP-1 peptide and/or other hormones in a subject in order to regulate blood sugar, weight, and hunger of the subject are described in this application. The compositions include a first nutritional supplement containing specific amounts of hydrolyzed yeast, acacia gum powder, citrus bioflavonoids, cinnamon bark, and eggplant whole vegetable. The compositions also include a second nutritional supplement containing specific amounts of medium chain triglycerides, resistant potato starch, resistant tapioca starch, kombucha, guar gum, blueberry juice powder, cranberry fruit powder, and grape seed extract. A third composition includes the first and second nutritional supplements containing these ingredients in sufficient amounts to regulate the GLP-1 hormone and/or other hormones. These compositions can increase the total GLP-1 production up to about 200%. Other embodiments are also described.

### CROSS-REFERENCE TO RELATED APPLICATION

This application is a continuation-in-part of U.S. application Ser. No. 18/910,816, filed Oct. 9, 2024, the entire disclosure of which is incorporated herein by reference.

### BACKGROUND

This application relates to compositions for activating the GLP-1 hormone in a subject. Specifically, this application describes nutritional supplements and compositions for activating the GLP-1 and other hormones in a subject in order to regulate blood sugar, weight, and hunger of the subject.

### SUMMARY

This application relates generally to nutritional supplements and compositions for activating the GLP-1 hormone and/or other hormones in a subject in order to regulate blood sugar, weight, and hunger of the subject. The compositions include a first nutritional supplement containing specific amounts of hydrolyzed yeast, acacia gum powder, citrus bioflavonoids, cinnamon bark, and eggplant whole vegetable. The compositions also include a second nutritional supplement containing specific amounts of medium chain triglycerides, resistant potato starch, resistant tapioca starch, kombucha, guar gum, blueberry juice powder, cranberry fruit powder, and grape seed extract. A third composition includes the first and second nutritional supplements containing these ingredients in sufficient amounts to regulate the GLP-1 hormone and/or other hormones. These compositions can increase the total GLP-1 production up to about 200%.

### BRIEF DESCRIPTION OF DRAWINGS

The following description can be better understood in light of the Figures that show various embodiments and configurations of the imaging systems and methods. Together with the following description, the Figures demonstrate and explain the structures, methods, and principles described herein. In the drawings, the thickness and size of components may be exaggerated or otherwise modified for clarity. The same reference numerals in different drawings represent the same element, and thus their descriptions will not be repeated. Furthermore, well-known structures, materials, or operations are not shown or described in detail to avoid obscuring aspects of the described devices and methods.

FIG. 1 illustrates the mechanism for creation of the GLP-1 hormone in the body from proglucagon.

FIG. 2 shows the mechanism of action of dipeptidyl peptidase-4 enzyme on GLP-1 to break down GLP-1 to an inactive form.

FIG. 3 shows the mechanism of creation of the PPY and PYY peptides.

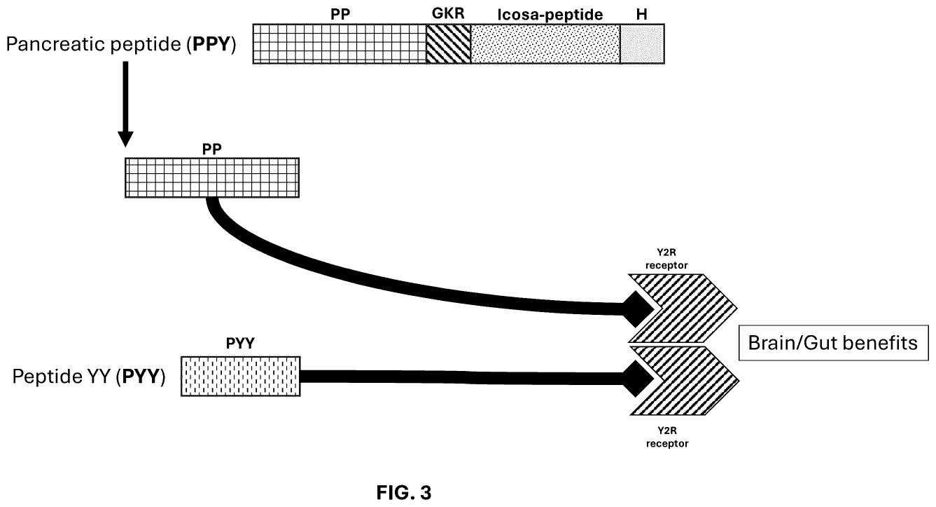

FIG. 4 shows an image of the GLP-1 hormone secreted in control cells not treated with any of the compositions described herein.

FIG. 5 shows an image of the GLP-1 hormone secreted in cells treated with the MBCore™ compositions described herein.

FIG. 6 shows an image of the GLP-1 hormone secreted in cells treated with the MindBody GLP-1 System™ compositions as described herein.

FIG. 7 depicts the in-vitro % change in mRNA GCG Gene Expression using the compositions described herein.

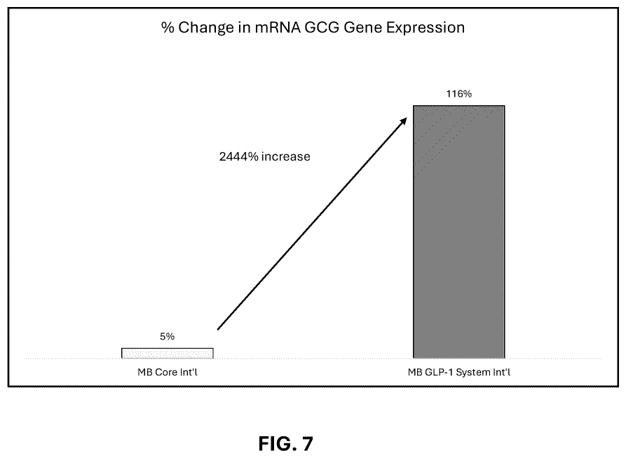

FIG. 8 depicts the in-vitro % change in mRNA DPP4 Gene Expression using the compositions described herein.

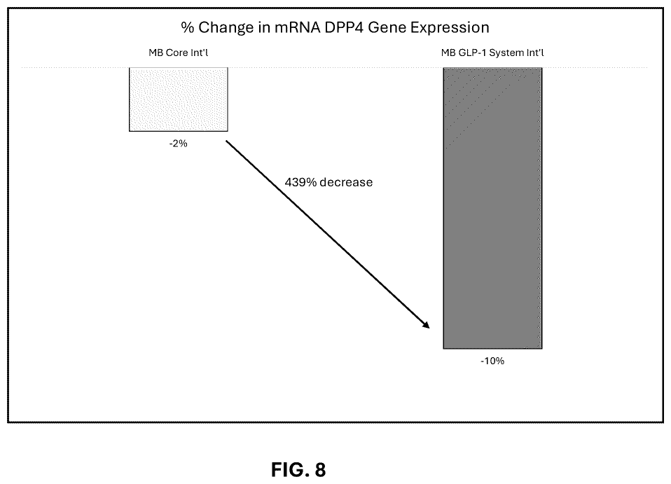

FIG. 9 shows the in-vitro % change in mRNA GLP1R Gene Expression using the compositions described herein.

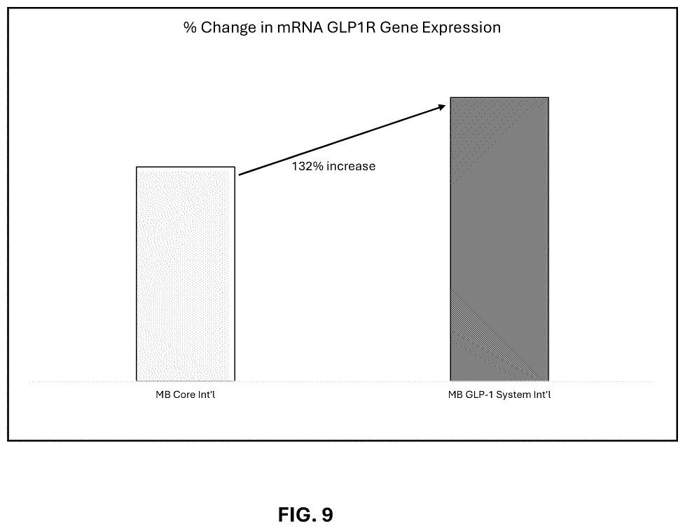

FIG. 10 depicts the in-vitro % change in mRNA PPY Gene Expression using the compositions described herein.

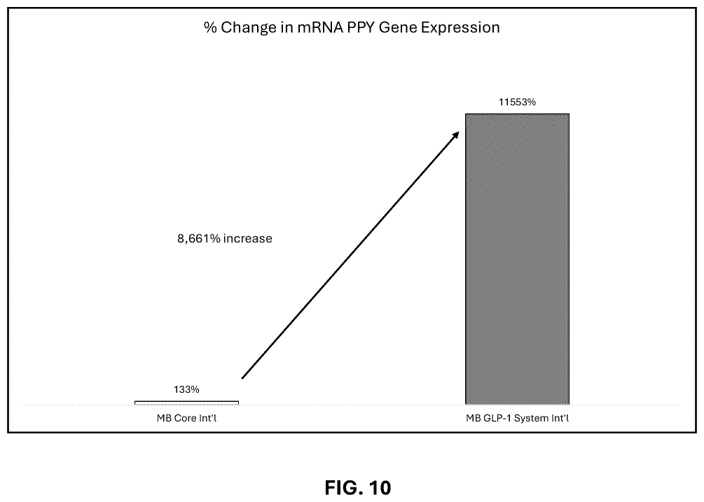

FIG. 11 depicts the in-vitro % change in mRNA PYY Gene Expression using the compositions described herein.

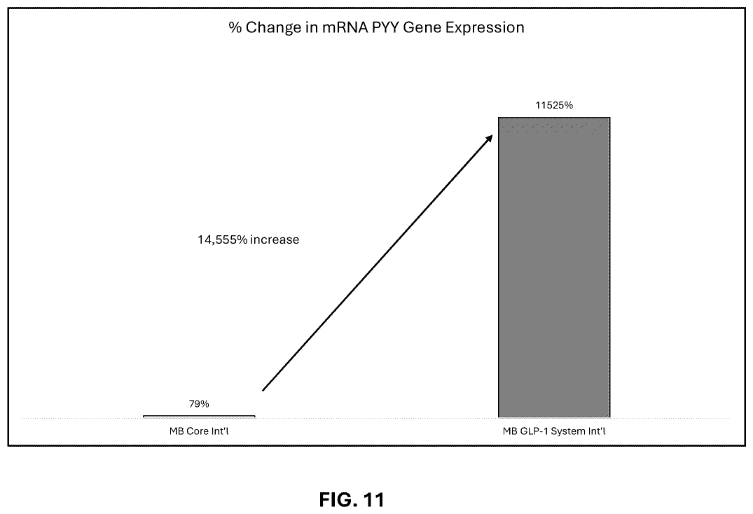

FIG. 12 shows the in-vitro % change in the GLP-1 production using the compositions described herein.

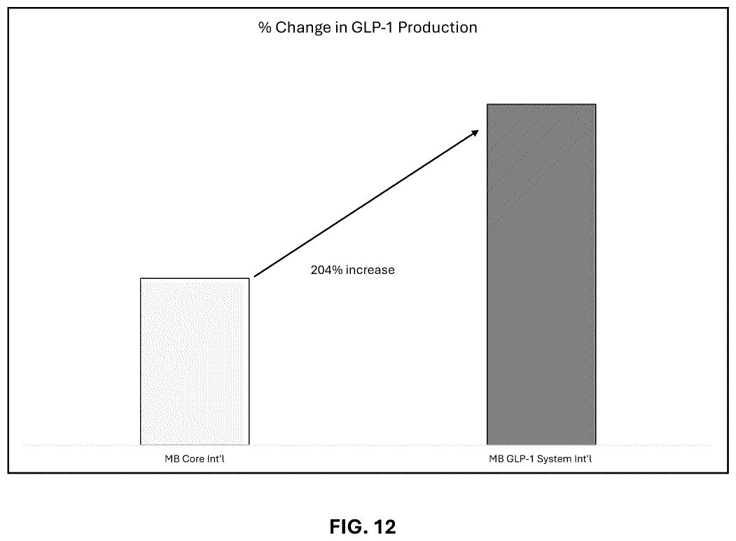

### DETAILED DESCRIPTION

The following description supplies specific details in order to provide a thorough understanding. Nevertheless, the skilled artisan will understand that the described methods and systems can be implemented and used without employing these specific details. Indeed, the described systems and methods can be placed into practice by modifying the described systems and methods and can be used in conjunction with any other apparatus and/or techniques conventionally used in the industry. For example, while the description below focuses on compositions and nutritional supplements and cosmetics for humans, it could be adapted and modified for other industries, such as animals or other uses such as being part of a functional food.

Food and the consumption of nutrients is important for individuals. Modern society has made it easy to gather and consume food and one of the downfalls is adding extra pounds to the human frame in the form of fat. The ease of obtaining excess food and the lack of physical exercise is contributing to the obesity epidemic that can be observed throughout the world. Our body has evolved to be able to take plant and animal components and convert them into nutrients that the body uses to elicit various chemical reactions that are pertinent to our survival.

Digestion of food involves both mechanical, microbial, and chemical processes. It can be divided into several stages that interact with one another. The first stage is the cephalic phase in the brain. The second stage is the gastric phase in the stomach. The third phase is the intestinal phase that is carried out in the small intestine and the colon.

These mechanisms require the organs to be as effective as possible and break down the nutrients in the food into meaningful cell signaling molecules. The digestive tract has evolved so that the cells in the digestive tract have developed highly specialized functions that are in constant state of communication. This communication can be in the form of direct connections via nerves, signaling throughout the body by hormones and other signaling molecules, and/or through an intricate network of internal cellular communication referred to as cellular signaling.

There are thousands of cellular signaling pathways adapting to changing environments within the body and they ultimately control the overall physiology of a body. For a signal to be transmitted within the cell, the correct signaling molecule must contact its corresponding receptor (like a lock and key). Once the lock turns or the receptor is activated, a chain of events happens throughout the cell with the end results being a specific response. This response can be for a cell to adapt to its environment and often happens on a micro-scale within a cell.

Once the receptors are activated, other signaling molecules can become activated, which can further activate other signaling molecules (and so on). The final signaling molecule's destination is ultimately the cell nucleus and the DNA, where genes (or information sequences) are turned on or off. These genes usually tell the cell what protein to make, such as insulin to shuttle glucose into the cell. The proteins are also responsible for making energy, hormones that ‘talk’ to the brain and stomach about appetite, and enzymes that defend the cell. All of these mechanisms working together make up the body's adaptive response to nutrients from food.

These cellular signaling pathways can be altered depending on the cellular environment in the body and the nutrients available from the food. As individuals become more sedentary and metabolic rates change, the cellular signaling pathways also become more sedentary and less efficient, which can lead to metabolic syndrome and insulin resistance.

Some of these cellular signaling pathways use enteroendocrine cells (EC). EC are specialized cells specifically for the gastrointestinal (GI) tract and pancreas. They have endocrine (hormone) function, and the GI tract is littered with many types of EC cells. The EC cells also release hormones or signaling peptides to initiate digestive actions or protective responses. Microbiome in the body also play a role in these responses because of their short chain fatty acid fermentation products that act as stimuli to some of the cells. Healthy digestion needs the function and interaction of all these cell types.

One specific type of EC used in food digestion is L-cells. L-cells are specialized enteroendocrine cells found primarily in the colon, to some degree in the small intestine, and in the pancreas. L-cells also help manage glucose and insulin levels and are stimulated by by-products from microbial fermentation in the colon to produce and secrete hormones such as glucagon-like peptide-1 (GLP-1), pancreatic peptide Y (PPY), and peptide YY (PYY). These three hormones have multiple paracrine and endocrine effects.

Upon food consumption, L-cells are stimulated to express the proglucagon gene (GCG) in several organs including the pancreas, gut, and brain. Depending on the organs, the proglucagon protein is further processed by tissue-specific posttranslational mechanisms to produce other peptides.

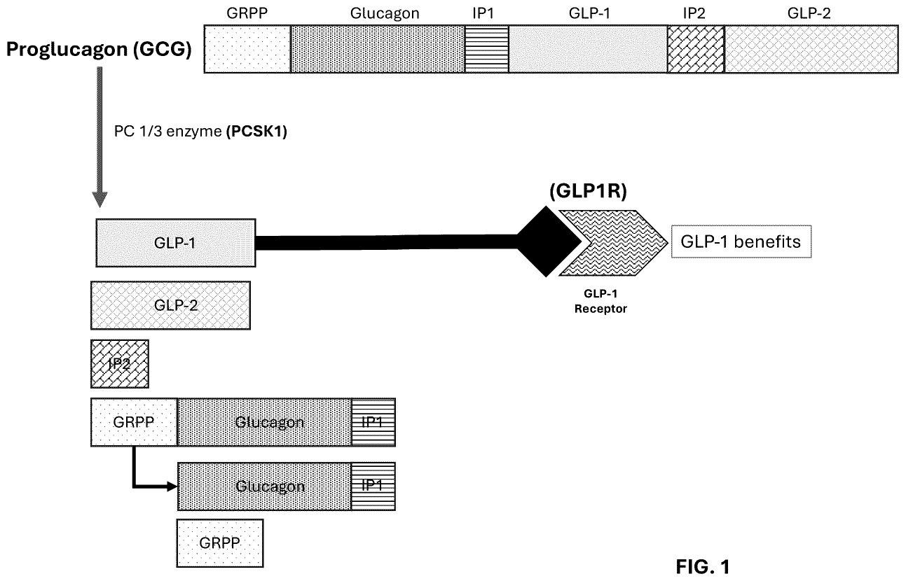

Pancreatic proglucagon can be stimulated by fasting and hypoglycemia and inhibited by insulin. It can be cleaved by prohormone convertase-2 enzyme (P/C 2) to produce glicentin-related pancreatic peptide (GRPP), glucagon, intervening peptide-1 (IP-1), and major proglucagon fragment (MPGF). Gut/brain proglucagon can also be stimulated by short-chain fatty acids, essential amino acids, sugars, and dietary fibers. As shown in FIG. 1, it can be post-translationally modified by prohormone convertase ⅓ (PC1/3) to produce glicentin-related pancreatic peptide (GRPP), oxyntomodulin, GLP-1, GLP-2, and intervening peptide-2 (IP-2).

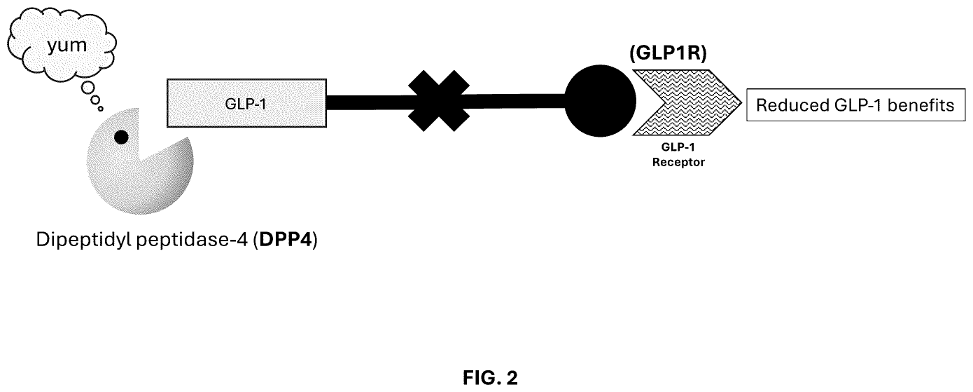

GLP-1 can be loaded into secretory vesicles within the cells and transported to the cell membrane where it is released into the hepatic portal system. Once GLP-1 is secreted, it is extremely susceptible to the proteolytic enzyme DPP-4 and only about 10-15% of secreted GLP-1 reaches the body's circulatory system. DPP-4 can also cleave GLP-1 to an inactive form, as shown in FIG. 2.

Another peptide produced by the L-cells is the peptide YY (PYY). It is closely related to the pancreatic peptide family and shows structural similarities to the pancreatic polypeptide (PPY) and neuropeptide Y (NPY). Two main forms of PYY (PYY₁₋₃₆ and PYY₃₋₃₆) are released by the L-cells upon nutrient stimulation. PYY 1-36 and PYY 3-36 differ in that the first two amino acids can be cleaved by the proteolytic DPP-4 enzyme at the N-terminal once secreted into the lumen. PYY₃₋₃₆ is highly selective to neuropeptide Y receptor type 2 (NPY2R) and is involved in the control of appetite, circadian rhythm, and anxiety.

Another peptide produced by the L-cells is pancreatic polypeptide Y (PPY). PPY is a polypeptide hormone secreted by the pancreas. It also is part of the neuropeptide Y family of peptides. Upon nutrient stimulation in the body, specialized cells in the pancreatic islets of Langerhans synthesize a pre-pro-pancreatic polypeptide which is further processed by specific posttranslational mechanisms to produce three peptides: pancreatic polypeptide (PPY), pancreatic icosapeptide, and a 5-7 amino acid-long oligopeptide. The peptides can then be secreted into the blood stream.

The GLP-1 hormone is part of the metabolic hormones called incretin. Incretins are released in response to nutrients, primarily glucose and fats and elicit an insulin response. GLP-1 has a short half-life of about 1-2 minutes once released into the hepatic portal system due to its breakdown by DPP-4. It can cross the blood/brain barrier by simple diffusion. Some GLP-1 is also produced in the brain. GLP-1 binds to the GLP-1 receptor GLP1R which is expressed in a variety of tissues such as pancreas, heart, kidneys, stomach, intestines, pituitary, hypothalamus, and Vagus nerve, thereby eliciting various chemical signaling processes. The GLP-1 hormone has several important functions. GLP-1 enhances the secretion of insulin from pancreatic beta-cells as a response to increased blood glucose levels and this enhances glucose-dependent insulin secretion. GLP-1 also suppresses glucagon secretion from pancreatic cells, further reducing glucose production in the liver. Further, GLP-1 delays gastric emptying of the stomach, which helps reduce postprandial blood glucose spikes. As well, GLP-1 interacts with central nervous system receptors to promote feelings of fullness and reducing appetite.

A related hormone, the PYY hormone, has an anorectic (loss of appetite) function in the body and plays a significant role in regulating appetite and food intake, including the following functions. PYY crosses the blood/brain barrier and directly interacts with the Y2 receptor in the brain. Through this interaction, it modulates the release of neurotransmitters involved in appetite regulation. PYY is released from the intestine in response to food intake, especially fats and proteins. It then binds to brain hypothalamus receptors and inhibits the activity of neuron PYY which is released from the intestine in response to nutrient intake, especially fats and proteins that stimulate appetite and promotes the activity of neurons that suppress appetite. This brings about a feeling of fullness and helps regulate food intake. PYY can inhibit the secretion of ghrelin in the stomach, which contributes to its appetite-suppressing effects. PYY helps slow down the emptying of the stomach, which then promotes a feeling of fullness after a meal. PYY influences gut motility, promoting digestion and longer absorption of nutrients while contributing to the feeling of satiety.

The PPY hormone is secreted after a protein meal, during fasting, exercise, and acute hypoglycemia and is decreased by somatostatin (produced in the stomach). It regulates pancreatic secretion activities through endocrine and exocrine tissue and overlaps in function with PYY. PPY works through the Vagus nerve, the nerve that connects the digestive tract to the brain, which allows it to send signals about hunger and satiety by the following mechanisms. PPY reduces the secretion of digestive enzymes and bicarbonate from the pancreas and thus regulates digestive processes. PPY acts on specific receptors in both the gut and brain, influencing the activity of other hormones and neurotransmitters involved in appetite and digestion. PPY signals the brain, especially the hypothalamus, to reduce food intake and appetite. PPY helps slow down the emptying of the stomach, which then promotes a feeling of fullness after a meal. PPY influences gut motility promoting digestion and longer absorption of nutrients while contributing to the feeling of satiety.

There are other hormones that work with GLP-1, PPY, and PYY to maintain balance and regulate food intake in the human body. As individuals consume food, signals from both hunger hormones and satiety hormones show an intricate play with the hypothalamus and other organs. Signals modulate food intake and energy expenditure to balance energy storage at the right levels for the right environmental conditions. Any change in this balance can lead to disorders, including type 2 diabetes, metabolic syndrome, and obesity.

When these hormones are out of balance, the conditions and disorders shown in Table 1 can occur.

TABLE 1

| Condition | Cause |
|---|---|
| Weight Gain | Increased levels of hunger hormones (like ghrelin) or decreased levels |
| | of satiety hormones (like leptin) can lead to overeating and weight gain. |
| Weight Loss | Conversely, excessive levels of satiety hormones or decreased hunger |
| | hormones can lead to reduced appetite and unintentional weight loss. |
| Metabolic | Imbalances can contribute to insulin resistance and increase the risk of |
| Disorders | type 2 diabetes. |
| Mood Changes | Hormonal imbalances can affect mood and lead to anxiety or |
| | depression, which can further impact eating behaviors. |
| Gastrointestinal | Altered hormone levels can affect digestion and gut motility, potentially |
| Issues | leading to issues like bloating or constipation. |
| Increased | Imbalances can lead to specific cravings, particularly for high-calorie or |
| Cravings | sugary foods. |
| Disrupted Eating | Hormonal imbalances may lead to irregular eating habits, such as binge |
| Patterns | eating or skipping meals. |

When the GLP-1 hormone is out of balance, it can lead to the various health issues shown in Table 2.

TABLE 2

| Condition | Cause |
|---|---|
| Insulin Resistance | Low levels of GLP-1 can contribute to insulin resistance, |
| | increasing the risk of type 2 diabetes. |
| Weight Gain | Insufficient GLP-1 can impair appetite regulation, leading to |
| | overeating and weight gain. |
| Glycemic Control Issues | Abnormal GLP-1 levels can disrupt glucose homeostasis, |
| | resulting in poor blood sugar control. |
| Gastrointestinal Problems | GLP-1 plays a role in gut motility; an imbalance may lead to |
| | digestive issues, such as slow gastric emptying or gastroparesis. |
| Increased Risk of | Since GLP-1 has protective cardiovascular effects, low levels |
| Cardiovascular Issues | may heighten the risk of heart disease. |

As evident from Table 2, low levels of GLP-1 hormone can contribute greatly to these health problems. There are several risk factors that can increase the risk of reduced GLP-1 production in the gut, but maintaining a healthy lifestyle can help support balanced GLP-1 production and levels. These risks factors include a hormonal imbalance, medications (especially those affecting glucose metabolism), gut health such as dysbiosis or other gastrointestinal issues, obesity with excess visceral fat, a diet low in fiber or high in fat, a sedentary lifestyle, type 2 diabetes, and aging.

One pathway to regulate the GLP-1 and associated hormones is through nutrigenomics. Nutrigenomics studies how foods and individual nutrients can affect gene expression and how genes can also affect food metabolism. Nutrigenomics has shown that nutrients can be added into the digestive system to basically reawaken those cellular signaling pathways.

The compositions described herein use the nutrigenomic mechanisms to impact expression of specific genes. They have been specifically formulated from nutrients in selected ratios to maximize the support of the body's natural cellular functions by targeting specific genetic and cellular signaling and biochemical pathways related to glucose response, appetite control, and fatty acid production by the microbiome. These compositions include the following three compositions: a MB Core™ composition, a MB Enhance™ composition, and a MindBody GLP-1System™ composition which combines the MB Core™ and MB Enhance™ compositions.

The first composition (MB Core™) was designed with a specific number of ingredients in the ratios that target L-cells throughout the intestinal tract. The MB Core™ composition stimulates GLP-1 production in L-cells and brings these cells to an optimum state of readiness when the body is in need of a healthy insulin/glucose response and influences energy homeostasis in the hypothalamus to regulate appetite. The second composition (MB Enhance™) was designed to target the microbiome in the colon. The microbiome is responsible for creating the correct types of fuel for L-cells. It does this by creating short-chain fatty acids from the resistant starches obtained from MB Enhance™ and thus optimizing the environment in the colon to facilitate microbial metabolism of butyrogenic bacteria. Both the MB Core™ and MB Enhance™ compositions were formulated to be combined into the MindBody GLP-1 System™ composition and regulate both gut specific L-cells and neuronal brain pathways involved in appetite regulation.

In some embodiments, the MB Core™ composition contains hydrolyzed yeast, acacia gum powder, citrus bioflavonoids, cinnamon bark, and eggplant (Solanum melongena L.) whole vegetable. In other embodiments, the MB Core™ composition can also contain microcrystalline cellulose and stearic acid.

The MB Core™ composition contains specific amounts of hydrolyzed yeast (Saccharomyces cervisciae) in some formulations. This ingredient can increase the production of the GLP-1 hormone and can activate appetite suppressing hormones. In some formulations, about 0.1 to about 5000 mg of this ingredient is present in the MB Core™ composition, or about 0.011 to about 84.11 wt %. In other formulations, about 50 to about 2000 mg of this ingredient is present in the MB Core™ composition, or about 5.025 to about 67.91 wt %. In yet other formulations, the MB Core™ composition contains about 34.6 wt % of this ingredient, or about 500 mg when the MB Core™ composition weighs about 1450 mg.

The MB Core™ composition contains specific amounts of acacia gum powder in some formulations. This ingredient is contained in the MB Core™ composition because it decreases the enzyme (DPP-4) that breaks down GLP-1. In some formulations, about 0.1 to about 5000 mg of this ingredient is present in the MB Core™ composition, or about 0.011 to about 84.11 wt %. In other formulations, about 50 to about 2000 mg of this ingredient is present in the MB Core™ composition, or about 5.025 to about 67.91 wt %. In yet other formulations, the MB Core™ composition contains about 34.6 wt % of this ingredient, or about 500 mg when the MB Core™ composition weighs about 1450 mg.

The MB Core™ composition also contains specific amounts of citrus bioflavonoids, including orange bioflavonoids, in some formulations. This ingredient is contained in the MB Core™ composition because it increases GLP-1 production in the blood. In some formulations, about 0.1 to about 2000 mg of this ingredient is present in the MB Core™ composition, or about 0.01 to about 65.7 wt %. In other formulations, about 50 to about 1000 mg of this ingredient is present in the MB Core™ composition, or about 4.57 to about 48.9 wt %. In yet other formulations, the MB Core™ composition contains about 27.7 wt % of this ingredient, or about 400 mg when the MB Core™ composition weighs about 1450 mg.

The MB Core™ composition can contain cinnamon bark in some formulations. This ingredient is contained in the MB Core™ composition because it supports insulin regulation. In some formulations, about 0.1 to about 500 mg of this ingredient is present in the MB Core™ composition, or up to about 25.84 wt %. In other formulations, about 1 to about 200 mg of this ingredient is present in the MB Core™ composition, or about 0.07 to about 12.23 wt %. In yet other formulations, the MB Core™ composition contains about 0.69 wt % of this ingredient, or about 10 mg when the MB Core™ composition weighs about 1450 mg.

The MB Core™ composition can also contain Eggplant (Solanum melongena L.) whole vegetable in some formulations. This ingredient is contained in the MB Core™ composition because it influences hunger hormones such as CCK, leptin, ghrelin, and neuropeptides. In some formulations, about 0.1 to about 1000 mg of this ingredient is present in the MB Core™ composition, or up to about 41.07 wt %. In other formulations, about 5 to about 500 mg of this ingredient is present in the MB Core™ composition, or about 0.347 to about 25.84 wt %. In yet other formulations, the MB Core™ composition contains about 0.69 wt % of this ingredient, or about 10 mg when the MB Core™ composition weighs about 1450 mg.

The MB Core™ composition can contain microcrystalline cellulose in some formulations. This ingredient is contained in the MB Core™ composition because it is an excipient and acts as a binder and flow agent. In some formulations, about 5 to about 500 mg of this ingredient is present in the MB Core™ composition or about 0.68 to about 25.47 wt %. In yet other formulations, the MB Core™ composition contains about 1.38 wt % of this ingredient, or about 20 mg when the MB Core™ composition weighs about 1450 mg.

The MB Core™ composition can contain stearic acid in some formulations. This ingredient is contained in the MB Core™ composition because it an excipient and act as a binder and flow agent. In some formulations, about 1 to about 50 mg of this ingredient is present in the MB Core™ composition or about 0.064 to about 39.12 wt %. In yet other formulations, the MB Core™ composition contains about 0.35 wt % of this ingredient, or about 5 mg when the MB Core™ composition weighs about 1450 mg.

With these ingredients, the MB Core™ composition can stimulate GLP-1 production in L-cells and brings these cells to an optimum state of readiness when the body is in need of a healthy insulin/glucose response and also influences energy homeostasis in the hypothalamus to regulate appetite. The MB Enhance™ composition was formulated to target the microbiome in the colon and enhance the function of the MB Core™ composition.

In some embodiments, the MB Enhance™ composition contains medium chain triglycerides (MCT), resistant potato starch, resistant tapioca starch, kombucha, guar gum, blueberry juice powder, cranberry fruit powder, grape seed extract, riboflavin, vitamin C, and zinc. The MB Enhance™ composition can also contain flavorings and excipients like sodium, citric acid, Stevia leaf extract, xanthan gum, and natural flavors.

The MB Enhance™ composition contains medium chain triglycerides (MCT). This ingredient is contained in the MB Enhance™ composition because it is a source for microbiome to produce short-chain fatty acids. In some formulations, about 0.1 to about 2000 mg of this ingredient is present in the MB Enhance™ composition, or up to about 10.02 wt %. In other formulations, about 50 to about 500 mg of this ingredient is present in the MB Enhance™ composition, or about 0.278 to about 2.71 wt %. In yet other formulations, the MB Enhance™ composition contains about 0.55 wt % of this ingredient, or about 100 mg when the MB Enhance™ composition weighs about 18 g.

The MB Enhance™ composition also contains resistant potato starch. This ingredient is contained in the MB Enhance™ composition because it is a resistant starch, an energy source for microbiome in colon. In some formulations, about 0.1 to about 20,000 mg of this ingredient is present in the MB Enhance™ composition, or up to about 71.25 wt %. In other formulations, about 1,000 to about 15,000 mg of this ingredient is present in the MB Enhance™ composition, or about 11.03 to about 65.02 wt %. In yet other formulations, the MB Enhance™ composition contains about 55 wt % of this ingredient, or about 10,000 mg when the MB Enhance™ composition weighs about 18g. In other formulations of the MB Enhance™ composition, any amount of resistant starch having a GLP-1 boosting effect can be added based on acceptability (minimal or no side effects like bloating) and cost.

The MB Enhance™ composition also contains resistant tapioca starch. This ingredient is contained in the MB Enhance™ composition because it is a resistant fiber, an energy source for microbiome in colon. In some formulations, about 0.1 to about 20,000 mg of this ingredient is present in the MB Enhance™ composition, or up to about 60.5 wt %. In other formulations, about 1,000 to about 15,000 mg of this ingredient is present in the MB Enhance™ composition, or about 7.1 to about 53.44 wt %. In yet other formulations, the MB Enhance™ composition contains about 27.67 wt % of this ingredient, or about 5000 mg when the MB Enhance™ composition weighs about 18g. In other formulations of the MB Enhance™ composition, any amount of resistant fiber having a GLP-1 boosting effect can be added based on acceptability (minimal or no side effects like bloating) and cost.

The MB Enhance™ composition also contains kombucha in some formulations. This ingredient is contained in the MB Enhance™ composition because it is an energy source for microbiome in the whole GI tract. In some formulations, about 0.1 to about 5000 mg of this ingredient is present in the MB Enhance™ composition, or up to about 22.06 wt %. In other formulations, about 5 to about 1000 mg of this ingredient is present in the MB Enhance™ composition, or about 0.028 to about 5.36 wt %. In yet other formulations, the MB Enhance™ composition contains about 2.21 wt % of this ingredient, or about 400 mg when the MB Enhance™ composition weighs about 18 g.

The MB Enhance™ composition can also contain guar gum in some formulations. This ingredient is contained in the MB Enhance™ composition because it is an energy source for microbiome in the whole GI tract. In some formulations, about 0.1 to about 2000 mg of this ingredient is present in the MB Enhance™ composition, or up to about 9.98 wt %. In other formulations, about 1 to about 500 mg of this ingredient is present in the MB Enhance™ composition, or about 0.0055 to about 2.7 wt %. In yet other formulations, the MB Enhance™ composition contains about 0.11 wt % of this ingredient, or about 20 mg when the MB Enhance™ composition weighs about 18 g.

The MB Enhance™ composition also contains blueberry juice powder in some formulations. This ingredient is contained in the MB Enhance™ composition because it is an energy source for microbiome in the whole GI tract. In some formulations, about 0.1 to about 5,000 mg of this ingredient is present in the MB Enhance™ composition, or up to about 22.06 wt %. In other formulations, about 5 to about 1000 mg of this ingredient is present in the MB Enhance™ composition, or about 0.028 to about 5.36 wt %. In yet other formulations, the MB Enhance™ composition contains about 2.2 wt % of this ingredient, or about 400 mg when the MB Enhance™ composition weighs about 18 g.

The MB Enhance™ composition also contains cranberry fruit powder in some formulations. This ingredient is contained in the MB Enhance™ composition because it is an energy source for microbiome in the whole GI tract. In some formulations, about 0.1 to about 5,000 mg of this ingredient is present in the MB Enhance™ composition, or up to about 22.06 wt %. In other formulations, about 5 to about 1000 mg of this ingredient is present in the MB Enhance™ composition, or about 0.028 to about 5.36 wt %. In yet other formulations, the MB Enhance™ composition contains about 2.2 wt % of this ingredient, or about 400 mg when the MB Enhance™ composition weighs about 18 g.

The MB Enhance™ composition also contains grape seed extract in some formulations. This ingredient is contained in the MB Enhance™ composition because it is an energy source for microbiome in the whole GI tract. In some formulations, about 0.1 to about 2,000 mg of this ingredient is present in the MB Enhance™ composition, or up to about 10.4 wt %. In other formulations, about 5 to about 500 mg of this ingredient is present in the MB Enhance™ composition, or about 0.028 to about 2.82 wt %. In yet other formulations, the MB Enhance™ composition contains about 0.69 wt % of this ingredient, or about 120 mg when the MB Enhance™ composition weighs about 18 g.

The MB Enhance™ composition can contain riboflavin in some formulations. This ingredient is contained in the MB Enhance™ composition because it is a vitamin and helps maintain good health. In some formulations, about 0.013 to about 130 mg of this ingredient is present in the MB Enhance™ composition, or up to about 1.1 wt %. In other formulations, about 0.026 to about 13 mg of this ingredient is present in the MB Enhance™ composition, or about 0.0002 to about 0.112 wt %. In yet other formulations, the MB Enhance™ composition contains about 0.0037 wt % of this ingredient, or about 0.43 mg when the MB Enhance™ composition weighs about 18 g.

The MB Enhance™ composition can also contain vitamin C in some formulations. This ingredient is contained in the MB Enhance™ composition because it is a vitamin and helps maintain good health. In some formulations, about 0.9 to about 7,000 mg of this ingredient is present in the MB Enhance™ composition, or from about 0.0062 wt % to about 32.5 wt %. In other formulations, about 1.8 to about 2000 mg of this ingredient is present in the MB Enhance™ composition, or about 0.012 to about 12.1 wt %. In yet other formulations, the MB Enhance™ composition contains about 0.42 wt % of this ingredient, or about 50 mg when the MB Enhance™ composition weighs about 18 g.

The MB Enhance™ composition can contain sodium in some formulations. This ingredient is contained in the MB Enhance™ composition because it adds flavor and as a mineral contributes to the general health of individuals. In some formulations, about 100 to about 1000 mg of this ingredient is present in the MB Enhance™ composition, or up to about 5 wt %. In other formulations, the MB Enhance™ composition contains about 2.82 wt % of this ingredient, or about 510 mg when the MB Enhance™ composition weighs about 18 g.

The MB Enhance™ composition can contain zinc in some formulations. This ingredient is contained in the MB Enhance™ composition because as a mineral, it contributes to the general health of individuals. In some formulations, about 0.1 to about 40 mg of this ingredient is present in the MB Enhance™ composition, or up to about 1.77 wt %. In other formulations, about 0.2 to about 33 mg of this ingredient is present in the MB Enhance™ composition, or about 0.009 to about 1.47 wt %. In yet other formulations, the MB Enhance™ composition contains about 0.180 wt % of this ingredient, or about 4 mg when the MB Enhance™ composition weighs about 18 g.

The MB Enhance™ composition can contain citric acid in some formulations. This ingredient is contained in the MB Enhance™ composition because it adds flavor. In some formulations, about 100 to about 2000 mg of this ingredient is present in the MB Enhance™ composition, or up to about 10 wt %. In other formulations, the MB Enhance™ composition contains about 3.06 wt % of this ingredient, or about 533 mg when the MB Enhance™ composition weighs about 18 g.

The MB Enhance™ composition can contain xanthan gum in some formulations. This ingredient is contained in the MB Enhance™ composition because it adds texture to the composition. In some formulations, about 50 to about 1,000 mg of this ingredient is present in the MB Enhance™ composition. In other formulations, the MB Enhance™ composition contains about 2 wt % of this ingredient, or about 360 mg when the MB Enhance™ composition weighs about 18 g.

The MB Enhance™ composition can contain stevia leaf extract in some embodiments. This ingredient is contained in the MB Enhance™ composition because it is a sweetener and adds flavor. In some formulations, about 0.1 to about 500 mg of this ingredient is present in the MB Enhance™ composition. In yet other formulations, the MB Enhance™ composition contains about 0.28 wt % of this ingredient, or about 51 mg when the MB Enhance™ composition weighs about 18 g.

The MB Enhance™ composition can also contain any mix of natural flavors in some formulations. In some formulations, about 0.1 to about 5,000 mg of this ingredient is present in the MB Enhance™ composition. In other formulations, the MB Enhance™ composition contains about 0.3 wt % of this ingredient, or about 55 mg when the MB Enhance™ composition weighs about 18 g.

In some embodiments, the following compounds that are GLP-1 receptor agonists could be used in place of, or in addition to, the GLP-1 receptor agonists that are used in the MB Core™ or MB Enhance™ compositions. These compounds include allulose, citrus fruits, Curcuma longa, oats, barley, legumes, omega-3 fatty acids, butyric acid, short-chain fatty acids, monounsaturated fats, green tea, Gymnema sylvestre, Trigonella foenum-graecum, bitter melon (Momordica charanita), aloe vera, mulberry, dandelion, milk thistle, Astragalus, Stevia rebaudiana, holy basil, hops, and/or combinations thereof.

In other embodiments, the following compounds that are DPP-4 receptor agonists could be used in place of, or in addition to, the DPP-4 receptor agonists that are used in the MB Core™ or MB Enhance™ compositions. These compounds include Trigonella foenum-graecum, green tea, mulberry, psyllium, allulose, Stevia rebaudiana, and/or combinations thereof.

In yet other embodiments, the following compounds that activate/reduce hypothalamus appetite genes/receptors could be used in place of, or in addition to, similar compounds that are used in the MB Core™ or MB Enhance™ compositions. These compounds include green tea, psyllium, Garcinia cambogia, Hoodia gordonii, Foeniculum vulgare (fennel), holy basil, slippery elm (Ulmus rubra), dandelion, Astragalus, cocoa, and/or combinations thereof.

The ingredients in the MB Core™ composition can be combined to create a single serving that can range from about 1 g to about 2 g. In some embodiments, the single serving can range from about 1.5 g to about 1.8 g. In other embodiments, the serving amount depends on the types of ingredients (active ingredient, stabilizers, excipients, and encapsulators) that are included in the composition. For example, when just active ingredients are included in the composition, the MB Core™ composition can be about 1.46 g or about 0.728 g when it is formulated in 2 capsules. In another example, when active ingredients and excipients are included in the composition, the MB Core™ composition can be about 1.56 g or about 0.782 g when it is formulated in 2 capsules. In yet another example, when active ingredients and a stabilizer are included in the composition, the MB Core™ composition can be about 1.46 g or about 0.728 g when it is formulated in 2 capsules. In yet another example, when active ingredients, excipients, and a stabilizer are included in the composition, the MB Core™ composition can be about 1.56 g or about 0.782 g when it is formulated in 2 capsules. In still another example, when active ingredients, excipients, a stabilizer, and an encapsulant are included in the composition, the MB Core™ composition can be about 1.82 g or about 0.908 g when it is formulated in 2 capsules.

The ingredients in the MB Enhance™ composition can be combined to create a single serving or 1 scoop that can range from about 14 to about 22 g. In some embodiments, the single serving or 1 scoop can range from about 16 to about 20 g. In other embodiments, the serving amount depends on the types of ingredients (flavorings, sweeteners) and overages that are included in the composition. For example, when both flavorings/sweeteners and overages are included in the MB Enhance™ composition, the total weight is about 18.069 g per 2 scoops or 9.034 g per 1 scoop for single serving. In another example, when only flavorings/sweeteners and no overages are included in the MB Enhance™ composition, the total weight is about 18.052 g per 2 scoops or 9.026 g per 1 scoop for single serving.

The MB Core™ and MB Enhance™ compositions can be prepared in various dosage forms. In some forms, they can be prepared as a capsule or a tablet. In other forms, they can be prepared as a powder. In some configurations, the MB Core™ composition is prepared as a capsule and the MB Enhance™ composition is prepared as a powder.

In some configurations, the MB Core™ composition can be formulated as follows:

In some configurations, the MB Enhance™ composition can be formulated as follows:

The MB Core™ and MB Enhance™ compositions can be used together or separately to help regulate weight loss for individuals and provide the other benefits described herein. In some embodiments, the MB Core™ and MB Enhance™ compositions can be combined to create a MindBody GLP-1 System™ composition that can be given to an individual at about the same time. When combined in these embodiments, the MindBody GLP-1 System™ composition contains the ingredients and the amounts shown in Table 3.

TABLE 3

| | Amount | Weight % |
|---|---|---|
| Ingredients | (mg) | in Blend |
| Yeast Hydrolysate | 500 | 2.805836% |
| Acacia gum | 500 | 2.805836% |
| Citrus bioflavonoids | 400 | 2.244669% |
| Medium Chain Triglycerides | 100 | 0.561167% |
| Resistant Potato Starch | 10,000 | 56.116723% |
| Resistant tapioca starch | 5,000 | 28.058361% |
| Kombucha | 400 | 2.244669% |
| Blueberry juice powder | 400 | 2.244669% |
| Cranberry juice powder | 400 | 2.244669% |
| Grape seed extract | 120 | 0.673401% |

In other embodiments, the MB Core™ and MB Enhance™ compositions can be taken separately within a 24-hour period even though better results come from consuming them at about the same time.

These amounts are based on 1.45 g of the MB Core™ composition being taken at about 30 minutes before the 18 g of the MB Enhance™ composition by an individual. When the relative amounts of the MB Core™ composition and MB Enhance™ composition taken are modified within the ranges described herein, the relative weight percentages of these ingredients will be modified as well.

### EXAMPLE: IN-VITRO

An in-vitro study was carried out to test the effect of the compositions described herein. Cell viability assays were performed for the MB Core™ and the MB Enhance™ composition to determine if NCI-H716 cells were impacted by their ingredients and to ascertain the optimal dosage for additional studies. The viability of NCI-H716 cells were evaluated using the Cell-Titer Glow Luminescent Cell assay based on quantification of ATP Adenosine triphosphate present, as an indicator of metabolically active cells. Cells were seeded at a density of 1000 cells/well in a 384-well white-walled cell culture plates and exposed at various concentrations (400, 200, 100, 50, 25, 12, 6, and 0 mcg/mL) to the MB Core™ and MB Enhance™ compositions the following day. The cells were allowed to grow in the presence of the MB Core™ and the MB Enhance™ compositions for 24 hours for dose-course studies. After this period, a Cell-Titer-Glo (Promega Corp., Madison WI) reagent was added at a volume equal to the cell culture medium in the plate according to manufacturer's instructions. The luminescence was read on an Envision 2105 multilabel reader (Perkin-Elmer, Waltham WI).

For gene expression studies and RNA extraction, the NCI-H716 cells were seeded at 0.5×10⁶ cells per well in 6-well tissue culture wells that had been treated with Matrigel. The cells were individually exposed to the MB Core™ composition alone, the MB Enhance™ composition alone, or the MindBody GLP-1 System™ composition about 72 hours after differentiation induced by the Matrigel. At indicated time points (3 hours, 8 hours, and 24 hours), RNA was extracted with the PureLink RNA Mini Kit (Thermo-Fisher Scientific, Waltham MA) using the instructions in this kit, with the exception that cells were lysed on the plate using 600 μL of lysis buffer. RNA was eluted with 45 μL of RNAse-free water and quantified on a NanoDrop spectrophotometer from Thermo-Fisher Scientific.

Reverse transcription/quantification real-time polymerase chain reaction (qRT-PCT) was then performed using the extracted RNA. A first-strand cDNA synthesis was carried out on 0.5 μg of the RNA using the SuperScript VILO Master Mix (Thermo-Fisher Scientific) following the manufacturer's instructions. A 6-fold dilution was made of each cDNA in PCR-grade water, and 6.5 μL of this solution was carried forward into qRT-PCR. The following PCR primers in Table 4 were purchased from Thermo Fisher Scientific and used.

TABLE 4

| Symbol | Identifier | Label |
|---|---|---|
| GCG | Hs01031536_m1 | FAM |
| PPY | Hs00358111_g1 | FAM |
| GLP1R | Hs00157705_m1 | FAM |
| DPP4 | Hs00897386_m1 | FAM |
| PCSK1 | Hs01026107_m1 | FAM |
| PYY | Hs00373890_g1 | FAM |
| ACTB | Hs01060665_g1 | VIC |
| | *endogenous control | |

The PCR reactions were carried out in 15 μL total volume of the following solution: 6.5 μL cDNA, 6 μL PCR-grade water, 0.5 μL gene-of-interest primer (FAM label), 0.5 μL actin primer (VIC label), and 7.5 μL Taqman Fast Advanced Master Mix (Thermo Fisher Scientific). The PCR reactions were performed using an Applied Biosystems QuantStudio Real-Time PCR Instrument (Thermo Fisher Scientific) under the following conditions: 50° C. for 2 minutes, 95° C. for 20 seconds, and then 40 cycles of 95° C. for 3 seconds and 60° C. for 30 seconds. The threshold cycle (CT) was determined using the instrument software. The differences in the threshold cycle between the gene of interest and actin (ACT) were determined for each sample and used to determine fold induction of each gene when compared to untreated controls.

The viability results were reported as follows. There was little to no difference in cell viability noted in the viability assays. It was unknown how close the highest dose of 400 μg/mL was to a dose limiting toxicity. In previous work with similar formulations, 400 μg/mL was considered on the high end of dosing.

The QPCR (Quantitative Polymerase Chain Reaction) results were reported as follows. The percent change observed for each gene target of interest using the MB Core™ and the MindBody GLP-1 System™ compositions are shown below in Table 5.

TABLE 5

| Gene target | Core | System |
|---|---|---|
| GCG (Proglucagon) | 5% | 116% |
| PCSK1 (PC 1/3 enzyme) | 0% | 0% |
| DPP4 (dipeptidyl dipeptidase 4) | −2% | −10% |
| GLP1R (GLP-1 receptor) | 41% | 55% |
| PPY (pancreatic polypeptide Y) | 133% | 11,553% |
| PYY (peptide YY) | 79% | 11,525% |

The results showed that there was both synergy and dual activation of specific genes using the MB Core™ and MB Enhance™ compositions. This result was measured by measured by the amplification/increase in the percent change int the gene expression. The gene activity of GCG, which codes for a pre-glucagon protein, increased by about 116% when treated with the MB GLP-1 System™ as compared to MB Core alone™ (about 5%): about a 2,444% increase. The same synergy was observed with DPP4 (about-2% to about-10%; about a 439% decrease), GLP1R (about 41% to about 55%; about a 132% increase), PPY (about 133% to about 11,553%; a surprising increase of about 8,661%), and PYY (about 79% to about 11,525%; a surprising increase of about 14,555%). The gene expression for the PCSK1 enzyme was not influenced significantly.

The amount of GLP-1 in-vitro secretion was also measured. The Human active GLP-1 (7-36) amide and GLP-1 (7-37) specific assay kit from Revvity with Homogeneous Time-Resolved Fluorescence (HTRF) technology was used to specifically look at active GLP-1 in the media and in the cells. The cells were treated with the MB Core™ composition, the MB Enhance™ composition, and a combination of the two. The assay was conducted according to manufacturer's specifications using an Envision 2105 multi-label reader that measured both 620 nm cryptate and 665 nm acceptor emissions after excitation with a 60 usec delay. The tissue culture media or cell lysate using RIPA buffer were used in the assay. Delta F ratios were calculated and used for interpolation of pro-collagen concentrations from a standard curve using any of the following normalization protocols: Prism; GraphPad; log (inhibitor) vs. response; or Variable slope.

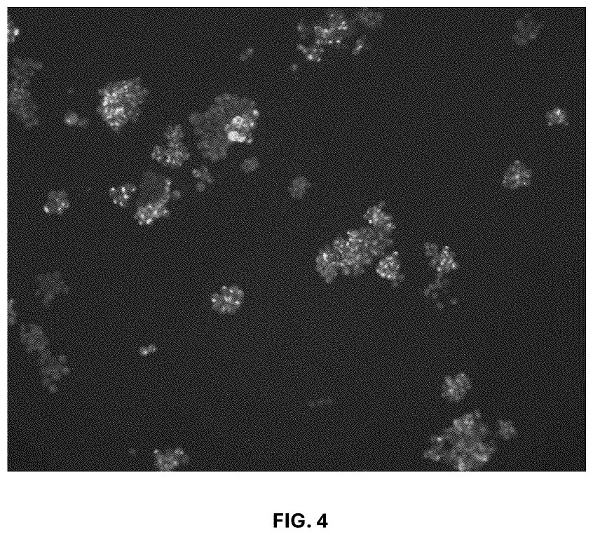

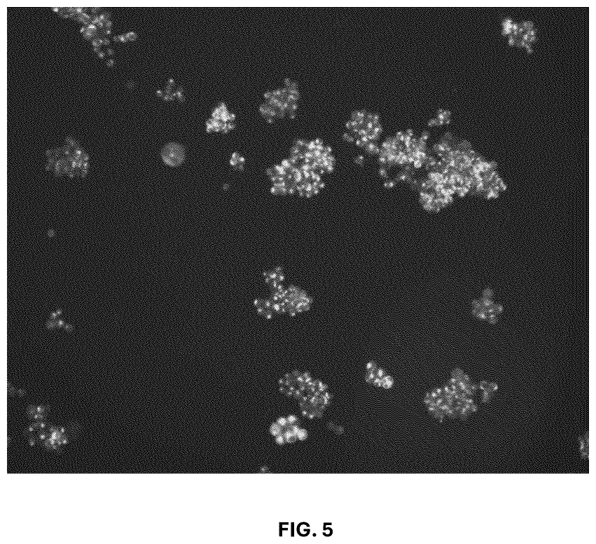

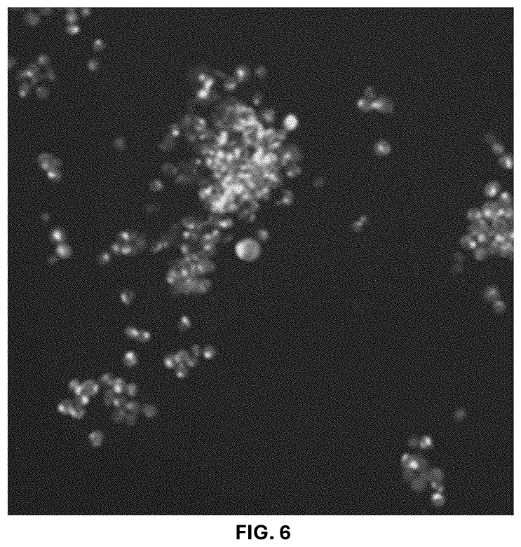

The total amount of GLP-1 protein secreted in both the cells and released into media showed a 122% increase after 24 hour incubation in the MindBody GLP-1 System™ composition. This was a 204% increase when compared to the MB Core™ composition alone. The amount of GLP-1 secreted was measured by the following imaging methods. NCI-H716 cells were grown in black 96-well plates with optically clear bottoms. The cells were fixed at appropriate time points (3 hours, 8 hours, and 24 hours) using 4% paraformaldehyde in PBS (phosphate-buffered saline) with a pH of 7.4 for 10 minutes at room temperature. Any fixed cells were washed three times in ice-cold PBS and then by permeabilization with PBS with 0.2% triton X-100. The cells were then washed and blocked with staining buffer containing 1% BSA (bovine serum albumin). The cells were then incubated in diluted Antibodies Online Cat. M2-42539 (1:100) in 1% BSA in PBST (PBS+1% Tween 20) in a humidified chamber for 1 hour. The cells were next incubated with secondary antibody Goat anti-rabbit AF488 (ThermoFisher Cat #3434-050-RTU) for 30 minutes. The cells were then counter-stained with DAPI 4′,6-diamino-2-phenylindole) for 15 minutes and washed once prior to imaging on an Evos FL auto 2 imager. The images showed more accumulation of GLP-1 protein inside cells when adding the MB Core™ composition, as shown in FIG. 5, or the MindBody GLP-1 System™ composition, as shown in FIG. 6, when compared to the control image, as shown in FIG. 4.

The average changes in gene expressions in this in vitro study using the MB Core™, MB Enhance™, and MindBody GLP-1 System™ compositions are summarized in Table 6.

TABLE 6

| | Negative | | | MindBody |
|---|---|---|---|---|
| | Control | MB | MB | GLP-1 |
| Gene | (Base) | Core ™ | Enhance ™ | System ™ |
| GCG | 1.003 ± | 1.050 ± | 1.343 ± | 2.163 ± |
| | 0.062 | 0.403 | 0.357 | 0.251 |
| PCSK1 | 1.051 ± | 1.076 ± | N/A | N/A |
| | 0.187 | 0.170 | | |
| DPP4 | 1.008 ± | 0.985 ± | 0.773 ± | 0.908 ± |
| | 0.173 | 0.109 | 0.143 | 0.222 |
| GLP1R | 1.042 ± | 1.474 ± | 1.338 ± | 1.615 ± |
| | 0.078 | 0.383 | 0.188 | 0.079 |
| PPY | 1.009 ± | 2.334 ± | 76.750 ± | 117.569 ± |
| | 0.024 | 0.246 | 12.954 | 19.333 |
| PYY | 1.009 ± | 1.807 ± | 77.690 ± | 119.435 ± |
| | 0.007 | 0.813 | 13.908 | 36.568 |

The average change in total GLP-1 production in this in vitro study using the MB Core™, MB Enhance™, and MindBody GLP-1 System™ compositions are shown in Table 7 and can range up to about 200%.

TABLE 7

| | Negative | | | MindBody |
|---|---|---|---|---|
| | Control | MB | MB | GLP-1 |
| | (Base) | Core ™ | Enhance ™ | System ™ |
| Total GLP-1 | 16.961 ± | 27.050 ± | 19.3560 ± | 37.585 ± |
| production | 0.805 | 2.358 | 0.450 | 0.476 |

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A nutritional supplement composition, comprising:
about 5 to about 68 wt % hydrolyzed yeast;
about 5 to about 68 wt % acacia gum powder;
about 4.6 to about 48.9 wt % citrus bioflavonoids;
about 0.07 to about 12 wt % cinnamon bark; and
about 0.35 to about 25.8 wt % eggplant whole vegetable.

2. The composition of claim 1, containing about 34.6 wt % hydrolyzed yeast.

3. The composition of claim 1, containing about 34.6 wt % acacia gum powder.

4. The composition of claim 1, containing about 27.7 wt % citrus bioflavonoids.

5. The composition of claim 1, containing about 0.7 wt % cinnamon bark.

6. The composition of claim 1, containing about 0.7 wt % eggplant whole vegetable.

7. A nutritional supplement composition, comprising:
about 0.28 to about 2.7 wt % medium chain triglycerides;
about 11 to about 65 wt % resistant potato starch;
about 7 to about 53.4 wt % resistant tapioca starch;
about 0.028 to about 5.36 wt % kombucha;
about 0.006 to about 2.7 wt % guar gum;
about 0.028 to about 5.4 wt % blueberry juice powder;
about 0.028 to about 5.4 wt % cranberry fruit powder; and
about 0.028 to about 2.8 wt % grape seed extract.

8. The composition of claim 7, containing about 0.55 wt % medium chain triglycerides.

9. The composition of claim 7, containing about 55.3 wt % resistant potato starch.

10. The composition of claim 7, containing about 27.7 wt % resistant tapioca starch.

11. The composition of claim 7, containing about 2.21 wt % kombucha.

12. The composition of claim 7, containing about 0.11 wt % guar gum.

13. The composition of claim 7, containing about 2.2 wt % blueberry juice powder.

14. The composition of claim 7, containing about 2.2 wt % cranberry fruit powder.

15. The composition of claim 7, containing about 0.69 wt % grape seed extract.

16. A nutritional supplement, comprising:
about 1 to about 2 g of a first composition comprising:
about 5 to about 68 wt % hydrolyzed yeast;
about 5 to about 68 wt % acacia gum powder;
about 4.6 to about 48.9 wt % citrus bioflavonoids;
about 0.07 to about 12 wt % cinnamon bark; and
about 0.35 to about 25.8 wt % eggplant whole vegetable. and about 14 to about 22 g of a second composition comprising:
about 0.28 to about 2.7 wt % medium chain triglycerides;
about 11 to about 65 wt % resistant potato starch;
about 7 to about 53.4 wt % resistant tapioca starch;
about 0.028 to about 5.36 wt % kombucha;
about 0.006 to about 2.7 wt % guar gum;
about 0.028 to about 5.4 wt % blueberry juice powder;
about 0.028 to about 5.4 wt % cranberry fruit powder; and
about 0.028 to about 2.8 wt % grape seed extract.

17. A nutritional supplement, comprising:
about 2.8 wt % hydrolyzed yeast;
about 2.8 wt % acacia gum powder;
about 2.24 wt % citrus bioflavonoids;
about 0.56 wt % medium chain triglycerides;
about 56.1 wt % resistant potato starch;
about 28.05 wt % resistant tapioca starch;
about 2.24 wt % kombucha;
about 2.24 wt % blueberry juice powder;
about 2.24 wt % cranberry fruit powder; and
about 0.67 wt % grape seed extract.

18. A method of activating GLP-1 and other hormones by administering a nutritional supplement composition to an individual, the composition comprising:
about 2.8 wt % hydrolyzed yeast;
about 2.8 wt % acacia gum powder;
about 2.24 wt % citrus bioflavonoids;
about 0.56 wt % medium chain triglycerides;
about 56.1 wt % resistant potato starch;
about 28.05 wt % resistant tapioca starch;
about 2.24 wt % kombucha;
about 2.24 wt % blueberry juice powder;
about 2.24 wt % cranberry fruit powder; and
about 0.67 wt % grape seed extract.

19. The method of claim 18, wherein the composition does not bind directly to the GLP-1 receptors.

20. The method of claim 18, wherein the average increase in total GLP-1 production using the composition ranges up to about 200%.

21. The method of claim 18, wherein administering the nutritional supplement composition to an individual resulted in a loss of body weight while maintaining or increasing the skeletal muscle.
